(19) United States
(12) Patent Application Publication
(10) Pub. No.: US 2003/0219863 A1
(43) Pub. Date: Nov. 27, 2003

(54) Soluble CTLA4 mutant molecules and uses thereof

(75) Inventors: Robert J. Peach, Southampton, PA (US); Joseph R. Naemura, Bellevue, WA (US); Peter S. Linsley, Seattle, WA (US); Jurgen Bajorath, Lynnwood, WA (US)

Correspondence Address: MANDEL & ADRIANO, 55 SOUTH LAKE AVENUE, SUITE 710, PASADENA, CA, 91101, US

(73) Assignee: Bristol-Myers Squibb Company

(21) Appl. No.: 10/336,384

(22) Filed: Jan. 2, 2003

Related U.S. Application Data
(63) Continuation of application No. 09/603,825, filed on Jun. 26, 2000, now abandoned.
Application No. 09/603,825 is a continuation-in-part of application No. 09/014,761, filed on Jan. 28, 1998, now abandoned.
(60) Provisional application No. 60/036,594, filed on Jan. 31, 1997.

(51) Int. Cl. A61K 38/17; C07H 21/04; C12P 21/02; C12N 5/06; C07K 14/74

(52) U.S. Cl. 435/69.1; 435/320.1; 435/325; 514/12; 530/350; 536/23.5

(57) ABSTRACT

The present invention provides soluble CTLA4 mutant molecules which bind with greater avidity to the CD80 and/or CD86 antigen than wildtype CTLA4 or non-mutated CTLA4Ig. The soluble CTLA4 molecules have a first amino acid sequence comprising the extracellular domain of CTLA4, where certain amino acid residues within the S25-R33 and M97-G107 are mutated. The mutant molecules of the invention also include a second amino acid sequence which increases the solubility of the mutant molecule.

This application is a continuation-in-part of U.S. Ser. No. 09/014,761, filed Jan. 28, 1998, which claims priority of U.S. Serial No. 60/036,549, filed Jan. 31, 1997, now abandoned, the contents of all of which are incorporated by reference into the present application.

Throughout this application various publications are referenced. The disclosures of these publications in their entireties are hereby incorporated by reference into this application in order to more fully describe the state of the art to which this invention pertains.

### FIELD OF THE INVENTION

The present invention relates to the field of soluble CTLA4 molecules which are mutated from wildtype to retain its ability to bind CD80 and/or CD86.

### BACKGROUND OF THE INVENTION

Antigen-nonspecific intercellular interactions between T-lymphocytes and antigen-presenting cells (APCs) generate T cell costimulatory signals that generate T cell responses to antigen (Jenkins and Johnson 1993 Curr. Opin. Immunol. 5:361-367). Costimulatory signals determine the magnitude of a T cell response to antigen, and whether this response activates or inactivates subsequent responses to antigen (Mueller et al. 1989 Annu. Rev. Immunol. 7: 445-480).

T cell activation in the absence of costimulation results in an aborted or anergic T cell response (Schwartz, R. H. 1992 Cell 71:1065-1068). One key costimulatory signal is provided by interaction of T cell surface receptors CD28 and CTLA4 with B7 related molecules on APC (e.g., also known as B7-1 and B7-2, or CD80 and CD86, respectively) (P. Linsley and J. Ledbetter 1993 Annu. Rev. Immunol. 11: 191-212).

The molecule now known as CD80 (B7-1) was originally described as a human B cell-associated activation antigen (Yokochi, T. et al. 1981 J. Immunol. 128:823-827; Freeman, G. J. et al. 1989 J. Immunol. 143:2714-2722), and subsequently identified as a counterreceptor for the related T cell molecules CD28 and CTLA4 (Linsley, P., et al. 1990 Proc. Natl. Acad. Sci. USA 87:5031-5035; Linsley, P. S. et al. 1991(a) J. Exp. Med. 173:721-730; Linsley, P. S. et al. 1991(b) J. Exp. Med. 174:561-570).

More recently, another counterreceptor for CTLA4 was identified on antigen presenting cells (APC) (Azuma, N. et al. 1993 Nature 366:76-79; Freeman 1993(a) Science 262:909-911; Freeman, G. J. et al. 1993(b) J. Exp. Med. 178:2185-2192; Hathcock, K. L. S., et al. 1994 J. Exp. Med. 180:631-640; Lenschow, D. J. et al., 1993 Proc. Natl. Acad. Sci. USA 90:11054-11058; Ravi-Wolf, Z., et al. 1993 Proc. Natl. Acad. Sci. USA 90:11182-11186; Wu, Y. et al. 1993 J. Exp. Med. 178:1789-1793).

This molecule, now known as CD86 (Caux, C., et al. 1994 J. Exp. Med. 180:1841-1848), but also called B7-0 (Azuma et al., 1993, supra) or B7-2 (Freeman et al., 1993a, supra), shares about 25% sequence identity with CD80 in its extracellular region (Azuma et al., 1993, supra; Freeman et al., 1993a, supra, 1993b, supra). CD86-transfected cells trigger CD28-mediated T cell responses (Azuma et al., 1993, supra; Freeman et al., 1993a, 1993b, supra).

Comparisons of expression of CD80 and CD86 have been the subject of several studies (Azuma et al. 1993, supra; Hathcock et al., 1994 supra; Larsen, C. P., et al. 1994 J. Immunol. 152:5208-5219; Stack, R. M., et al., 1994 J. Immunol. 152:5723-5733). Current data indicate that expression of CD80 and CD86 are regulated differently, and suggest that CD86 expression tends to precede CD80 expression during an immune response.

Soluble forms of CD28 and CTLA4 have been constructed by fusing variable (v)-like extracellular domains of CD28 and CTLA4 to immunoglobulin (Ig) constant domains resulting in CD28Ig and CTLA4Ig. CTLA4Ig binds both CD80 positive and CD86 positive cells more strongly than CD28 μg (Linsley, P. et al. 1994 Immunity 1:793-80). Many T cell-dependent immune responses are blocked by CTLA4Ig both in vitro and in vivo. (Linsley, et al., (1991b), supra; Linsley, P. S. et al., 1992(a) Science 257:792-795; Linsley, P. S. et al., 1992(b) J. Exp. Med. 176:1595-1604; Lenschow, D. J. et al. 1992, Science 257:789-792; Tan, P. et al., 1992 J. Exp. Med. 177:165-173; Turka, L. A., 1992 Proc. Natl. Acad. Sci. USA 89:11102-11105).

Peach et al., (J. Exp. Med. (1994) 180:2049-2058) identified regions in the CTLA4 extracellular domain which are important for strong binding to CD80. Specifically, a hexapeptide motif (MYPPPY) in the complementarity determining region 3 (CDR3)-like region was identified as fully conserved in all CD28 and CTLA4 family members. Methionine scanning mutagenesis through the motif in CTLA4 and at selected residues in CD28Ig reduced or abolished binding to CD80.

Chimeric molecules interchanging homologous regions of CTLA4 and CD28 were also constructed. Molecules HS4, HS4-A and HS4-B were constructed by grafting CDR3-like regions of CTLA4 which also included a portion carboxy terminally extended to include certain nonconserved amino acid residues onto CD28Ig. These homologue mutants showed higher binding avidity to CD80 than did CD28Ig.

In another group of chimeric homologue mutants, the CDR1-like region of CTLA4, which is not conserved in CD28 and is predicted to be spatially adjacent to the CDR3-like region was grafted, into HS4 and HS4-A. These chimeric homologue mutant molecules (designated HS7 and HS8) demonstrated even greater binding avidity for CD80.

Chimeric homologue mutant molecules were also made by grafting into HS7 and HS8 the CDR2-like region of CTLA4, but this combination did not further improve the binding avidity for CD80. Thus, the MYPPPY motif of CTLA4 and CD28 were determined to be critical for binding to CD80, but certain non-conserved amino acid residues in the CDR1- and CDR3-like regions of CTLA4 were also responsible for increased binding avidity of CTLA4 with CD80.

CTLA4Ig was shown to effectively block CD80-associated T cell co-stimulation but was not as effective at blocking CD86-associated responses. Soluble CTLA4 mutant molecules, especially those having a higher avidity for CD86 than wild type CTLA4, were constructed as possibly better able to block the priming of antigen specific activated cells than CTLA4Ig.

Site-directed mutagenesis and a novel screening procedure were used to identify several mutations in the extracellular domain of CTLA4. The resulting mutants retained their ability to bind CD80 and/or CD86 and in some cases exhibited improved binding avidity for CD80 and/or CD86 as compared to wildtype. These molecules will provide better pharmaceutical compositions for immune suppression and cancer treatment than previously known soluble forms of CTLA4.

### SUMMARY OF THE INVENTION

The invention provides soluble CTLA4 mutant molecules that bind CD80 and/or CD86. Mutant molecules of the invention include those that can recognize and bind either of CD80, CD86, or both. In some embodiments, some mutant molecules bind CD80 and/or CD86 with greater avidity than CTLA4.

Examples of CTLA4 mutant molecules include L104EA29YIg (FIG. 7). The amino acid sequence of L104EA29YIg can begin at alanine at amino acid position −1 and end at lysine at amino acid position +357. Alternatively, the amino acid sequence of L104EA29YIg can begin at methionine at amino acid position +1 and end at lysine at amino acid position +357. The CTLA4 portion of L104EA29YIg encompasses methionine at amino acid position +1 through aspartic acid at amino acid position +124. L104EA29YIg comprises a junction amino acid residue glutamine at position +125 and an immunoglobulin portion encompassing glutamic acid at position +126 through lysine at position +357 (FIGS. 7 and 8). L104EA29YIg binds approximately 2-fold more avidly than wildtype CTLA4Ig (hereinafter referred to as CTLA4Ig) to CD80 and 4-fold more avidly to CD86. This stronger binding results in L104EA29YIg being up to 10-fold more effective than CTLA4Ig at blocking immune responses.

Another example of a CTLA4 mutant molecule is L104EIg (FIG. 8). L104EIg also binds CD80 and CD86 more avidly than CTLA4Ig.

### BRIEF DESCRIPTION OF THE FIGURES

FIG. 1: Equilibrium binding analysis of L104EA29YIg, L104EIg, and wild-type CTLA4Ig to CD86Ig. L104EA29YIg binds more strongly to CD86Ig than does L104EIg or CTLA4Ig. Equilibrium binding constants (Kd) were determined and shown in Table 1 (Example 2). The lower Kd of L104EA29YIg (3.21) than L104EIg (6.06) or CTLA4Ig (13.9) indicates higher binding avidity to CD86Ig. The lower Kd of L104EA29YIg (3.66) than L104EIg (4.47) or CTLA4Ig (6.51) indicates higher binding avidity to CD80Ig.

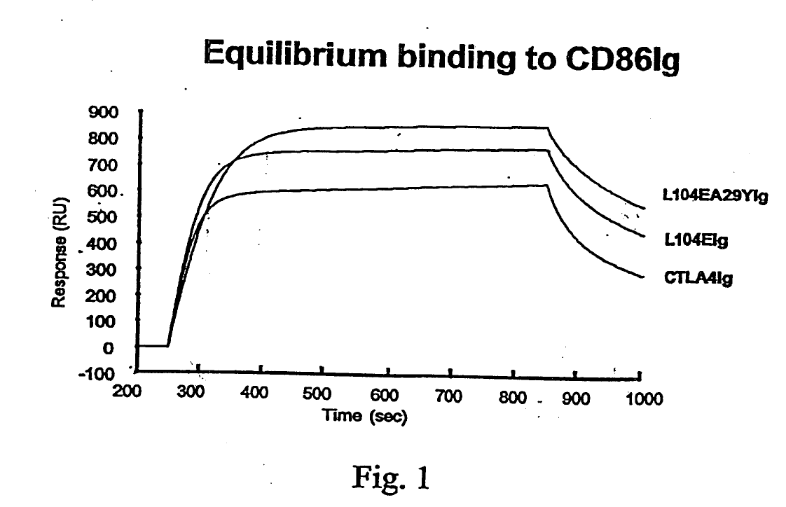

FIG. 2: FACS assay showing L104EA29YIg and L104EIg bind more strongly to CHO cells stably transfected with human CD86 than does CTLA4Ig. Binding of each protein to human CD80-transfected CHO cells appears to be equivalent.

FIG. 3: In vitro functional assays showing that L104EA29YIg is ˜10-fold more effective than CTLA4Ig at inhibiting proliferation of CD86+ PMA treated human T cells. Inhibition of CD80+ PMA stimulated proliferation by CTLA4Ig and L104EA29YIg is more equivalent.

FIG. 4: L104EA29YIg is approximately 10-fold more effective than CTLA4Ig at inhibiting proliferation of primary and secondary allostimulated T cells. A) The effect of L103EA29YIg on primary allostimulated T cells. B) The effect of L103EA29YIg on secondary allostimulated T cells.

FIG. 5: L104EA29YIg is 5-7-fold more effective than CTLA4Ig at inhibiting IL-2, IL-4, and γ-interferon cytokine production of allostimulated human T cells.

FIG. 6: L104EA29YIg is ˜10-fold more effective than CTLA4Ig at inhibiting proliferation of PHA-stimulated monkey PBMC's.

FIG. 7: Depicts the nucleotide and amino acid sequence of L104EA29YIg starting at methionine at position +1 to aspartic acid at position +124, or alanine at position −1 to aspartic acid at position +124.

FIG. 8: Depicts the nucleotide and amino acid sequences of L104EIg starting at methionine at position +1 to aspartic acid at position +124, or alanine at position −1 to aspartic acid at position +124.

FIG. 9: Depicts the amino acid sequence of a CTLA4Ig having wildtype extracellular domain of CTLA4.

FIG. 10: Depicts the full-length amino acid sequence of the immature form of naturally occurring CTLA4.

FIG. 11: Depicts a nucleotide and amino acid sequence of a soluble CTLA4 mutant molecule, comprising a signal peptide, the mutated extracellular domain of CTLA4, and Ig region.

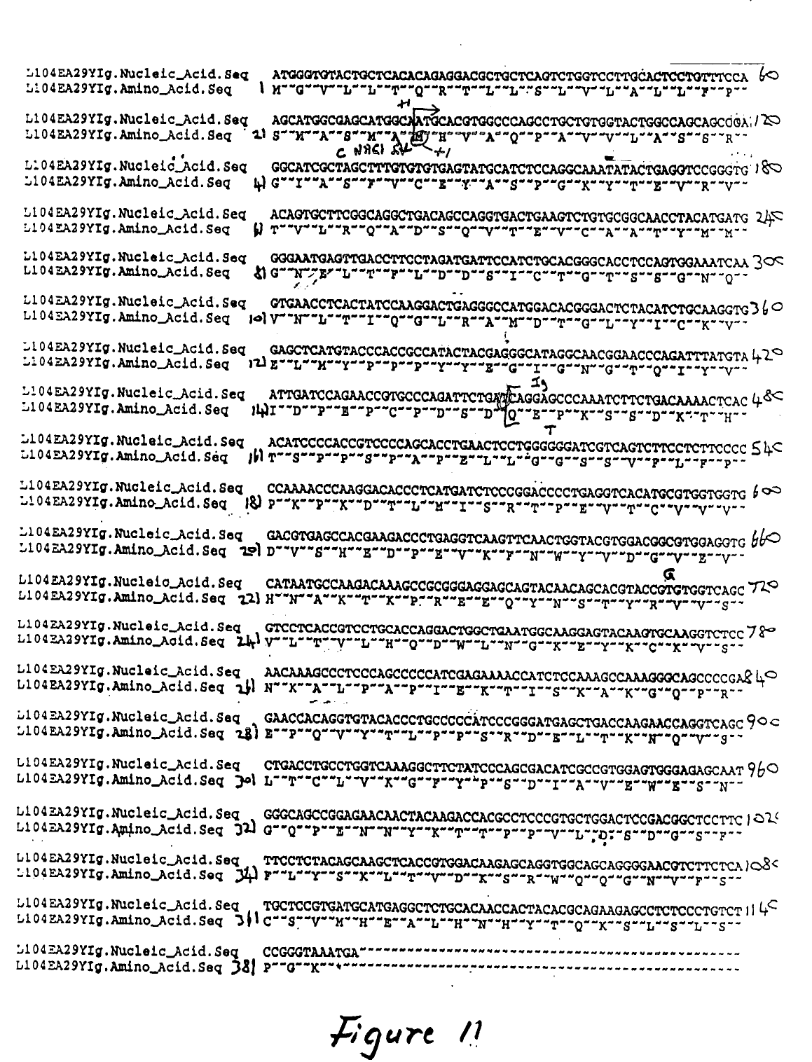

FIG. 12: Depicts a nucleotide and amino acid sequence of a soluble CTLA4 mutant molecule, comprising a signal peptide, the mutated extracellular domain of CTLA4, and Ig region.

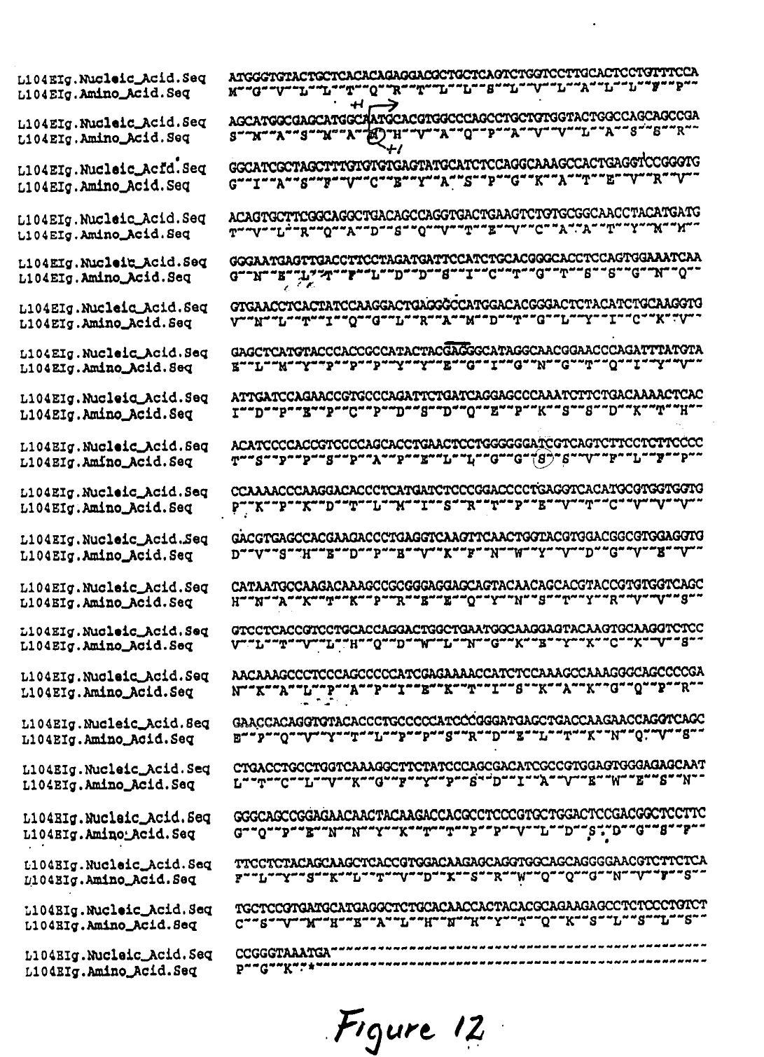

### DETAILED DESCRIPTION OF THE INVENTION

Definitions

As used in this application, the following words or phrases have the meanings specified.

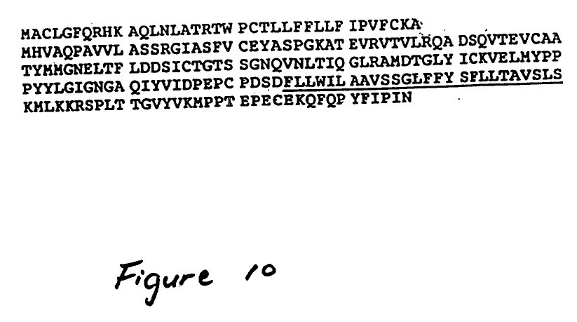

As used herein “wildtype CTLA4” has the sequence of naturally occurring, full length CTLA4 (FIG. 10) and U.S. Pat. Nos. 5,434,131, 5,844,095, 5,851,795, or any portion thereof which binds CD28 and/or CD86 or interferes with CD80 and/or CD86 so that it blocks its binding to its ligand, or the extracellular domain of CTLA4 of portions thereof. CTLA4 is a cell surface protein, having an N-terminal extracellular domain, a transmembrane domain, and a C-terminal cytoplasmic domain. The extracellular domain binds to target antigens, such as CD80 and CD86. In a cell, the naturally occurring, wild type CTLA4 protein is translated as an immature polypeptide, which includes a signal peptide at the N-terminal end. The immature polypeptide undergoes post-translational processing, which includes cleavage and removal of the signal peptide to generate a CTLA4 cleavage product having a newly generated N-terminal end that differs from the N-terminal end in the immature form. One skilled in the art will appreciate that additional post-translational processing may occur, which removes one or more of the amino acids from the newly generated N-terminal end of the CTLA4 cleavage product. The mature form of the CTLA4 molecule includes the extracellular domain or any portion thereof which binds to CD80 and/or CD86.

As used herein a “CTLA4 mutant molecule” is a molecule can be full length CTLA4 or portions thereof (derivatives or fragments) that have a mutation or multiple mutations in the extracellular domain of CTLA4 has been made so that it is similar but no longer identical to the wildtype CTLA4 molecule. Mutant CTLA4 molecules may include a biologically or chemically active non-CTLA4 molecule therein or attached thereto. The mutant molecules may be soluble (i.e., circulating) or bound to a surface. CTLA4 mutant molecules can include the entire extracellular domain of CTLA4 (CTLA4 mutant molecules can be made synthetically or recombinantly) or portions thereof, e.g., fragments or derivatives.

As used herein, the term “mutation” means a change in the amino acid sequence of the wildtype CTLA4 extracellular domain. The amino acid changes include substitutions, deletions, additions, or truncations. The mutant molecule can have one or more mutations.

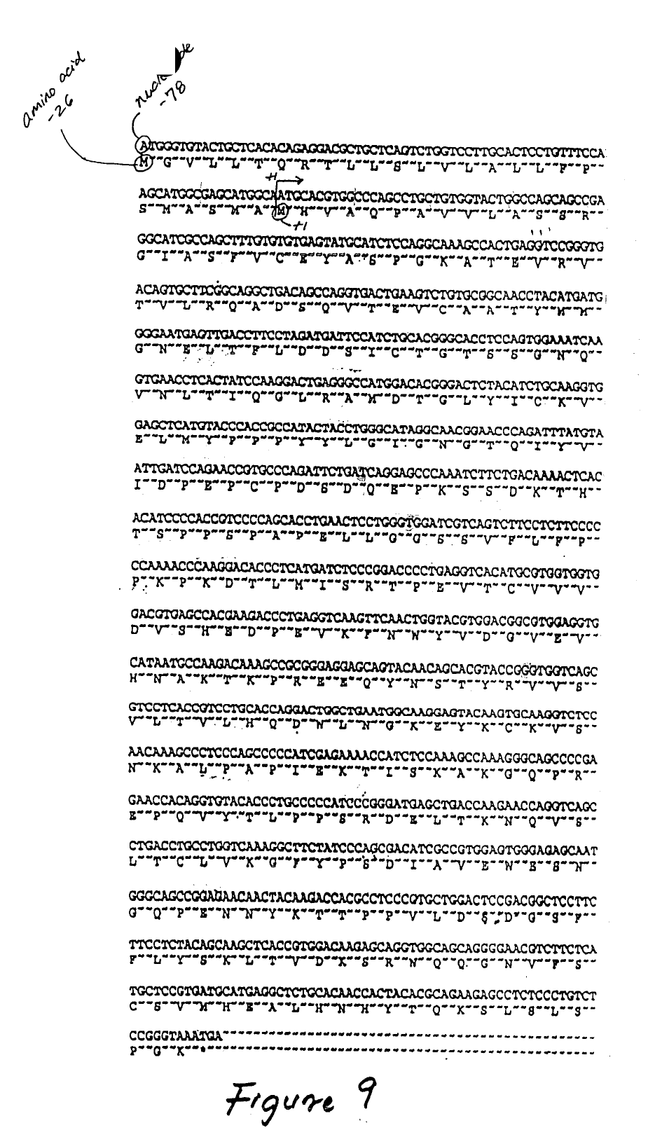

As used herein “the extracellular domain of CTLA4” is a portion of CTLA4 which recognizes and binds CD80 and/or CD86. For example, an extracellular domain of CTLA4 comprises methionine at position +1 to aspartic acid at position +124 (FIG. 9). Alternatively, an extracellular domain of CTLA4 comprises alanine at position −1 to aspartic acid at position +124 (FIG. 9). The extracellular domain includes fragments or derivatives of CTLA4 that bind CD80 and/or CD86.

As used herein a “non-CTLA4 protein sequence” or “non-CTLA4 molecule” means any protein molecule which does not bind CD80 and/or CD86 and does not interfere with the binding of CTLA4 to its target. An example includes, but is not limited to, an immunoglobulin (Ig) constant region or portion thereof. Preferably, the Ig constant region is a human or monkey Ig constant region, e.g., human C(gamma)1, including the hinge, CH2 and CH3 regions. The Ig constant region can be mutated to reduce its effector functions (U.S. Pat. Nos. 5,844,095; 5,851,795; and 5,885,796).

As used herein a “fragment of CTLA4 mutant molecule” is any portion of CTLA4 mutant molecule, preferably the extracellular domain of CTLA4 or a portion thereof that recognizes and binds its target, e.g., CD80 and/or CD86.

As used herein a “derivative of CTLA4 mutant molecule” is a molecule that shares at least 70% sequence similarity with and functions like the extracellular domain of CTLA4, i.e., it recognizes and binds CD80 and/or CD86.

In order that the invention herein described may be more fully understood, the following description is set forth.

Compositions of the Invention

The present invention provides soluble CTLA4 mutant molecules which recognize and bind CD80 and/or CD86. In some embodiments, the soluble CTLA4 mutants have a higher avidity to CD80 and/or CD86 than CTLA4Ig, because they should be better able to interfere or disrupt the priming of antigen specific activated cells than CTLA4Ig.

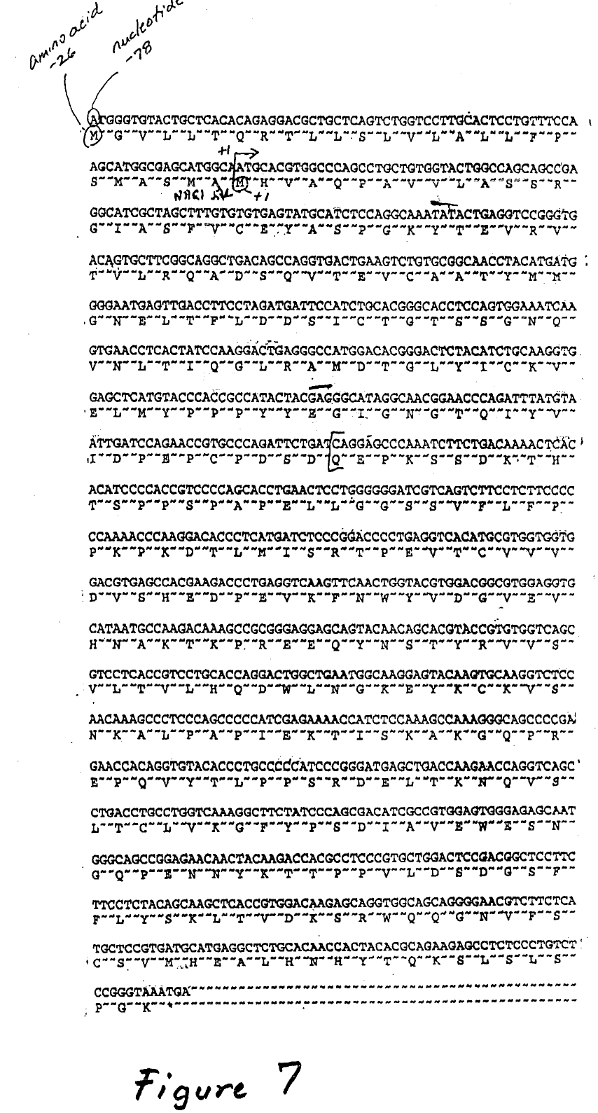

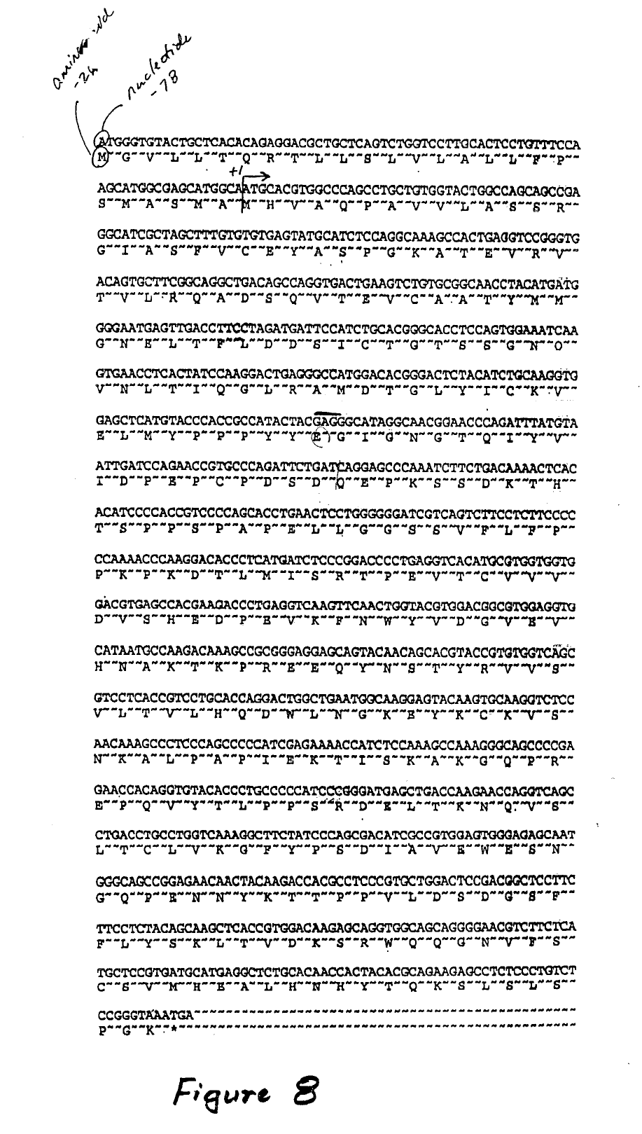

CTLA4 mutant molecules comprise at least the extracellular domain of CTLA4 or portions thereof that bind CD80 and/or CD86. The extracellular portion of CTLA4 comprises methionine at position +1 through aspartic acid at position +124 (FIG. 7 or 8). Alternatively, the extracellular portion of the CTLA4 can comprise alanine at position −1 through aspartic acid at position +124 (FIG. 7 or 8).

In one embodiment, the soluble CTLA4 mutant molecule is a fusion protein comprising the extracellular domain of CTLA4 having one or more mutations in a region beginning with serine at +25 and ending with arginine at +33 (S25-R33). For example, the alanine at position +29 of wildtype CTLA4 can be substituted with tyrosine (codons: UAU, UAC). Alternatively, alanine can be substituted with leucine (codons: UUA, UUG, CUU, CUC, CUA, CUG), phenylalanine (codons: UUU, UUC), tryptophan (codon: UGG), or threonine (codons: ACU, ACC, ACA, ACG).

In another embodiment, the soluble CTLA4 mutant molecule is a fusion protein comprising the extracellular domain of CTLA4 having one or more mutations in or near a region beginning with methionine at +97 and ending with glycine at +107 (M97-G107). For example, leucine at position +104 of wildtype CTLA4 can be substituted with glutamic acid (codons: GAA, GAG). A CTLA4 mutant molecule having this substitution is referred to herein as L104EIg (FIG. 8).

In yet another embodiment, the soluble CTLA4 mutant molecule is a fusion protein comprising the extracellular domain of CTLA4 having one or more mutations in the S25-R33 and M97-G107. For example, in one embodiment, a CTLA4 mutant molecule comprises tyrosine at position +29 instead of alanine; and glutamic acid at position +104 instead of leucine. A CTLA4 mutant molecule having these substitutions is referred to herein as L104EA29YIg (FIG. 7). (ATCC No. Not Yet Assigned) This nucleic acid molecule encodes L104EA29YIg, was deposited on Jun. 19, 2000 with the American Type Culture Collection (ATCC), 10801 University Blvd., Manasas, Va. 20110-2209.

The invention further provides a soluble CTLA4 mutant molecule comprising an extracellular domain of CTLA4 mutant as shown in FIG. 7 or 8 or portion(s) thereof and a moiety that alters the solubility, affinity and/or valency of the CTLA4 mutant molecule for binding CD80 and/or CD86.

In accordance with a practice of the invention, the moiety can be an immunoglobulin constant region or portion thereof. For in vivo use, it is preferred that the immunoglobulin constant region does not elicit a detrimental immune response in the subject. For example, in clinical protocols, it may be preferred that mutant molecules include human or monkey immunoglobulin constant regions. One example of a suitable immunoglobulin region is human C(gamma)1, comprising the hinge, CH2, and CH3 regions. Other isotypes are possible. Further, other immunoglobulin constant regions are possible (preferably other weakly or non-immunogenic immunoglobulin constant regions).

Other moieties include polypeptide tags. Examples of suitable tags include but are not limited to p97 molecule, env gp120-molecule, E7 molecule, and ova molecule (Dash, B., et al. 1994 J. Gen. Virol. 75:1389-97; Ikeda, T., et al. 1994 Gene 138:193-6; Falk, K., et al. 1993 Cell. Immunol 150:447-52; Fujisaka, K. et al. 1994 Virology 204:789-93). Other molecules are possible (Gerard, C. et al. 1994 Neuroscience 62:721; Byrn, R. et al. 1989 63:4370; Smith, D. et al., 1987 Science 238:1704; Lasky, L., 1996 Science 233:209).

The invention further provides soluble mutant CTLA4Ig fusion proteins preferentially more reactive with the CD80 and/or CD86 antigen compared to wildtype CTLA4. One example is L104EA29YIg as shown in FIG. 7.

In another embodiment, the soluble CTLA4 mutant molecule includes a junction amino acid residue which is located between the CTLA4 portion and the immunoglobulin portion. The junction amino acid can be any amino acid, including glutamine. The junction amino acid can be introduced by molecular or chemical synthesis methods known in the art.

In another embodiment, the soluble CTLA4 mutant molecule includes the immunoglobulin portion (e.g., hinge, CH2 and CH3 domains), where any or all of the cysteine residues, within the hinge domain are substituted with serine, for example, the cysteines at positions +130, +136, or +139 (FIG. 7 or 8). The mutant molecule may also include the proline at position +148 substituted with a serine, as shown in FIG. 7 or 8.

The soluble CTLA4 mutant molecule can include a signal peptide sequence linked to the N-terminal end of the extracellular domain of the CTLA4 portion of the mutant molecule. The signal peptide can be any sequence that will permit secretion of the mutant molecule, including the signal peptide from oncostatin M (Malik, et al., 1989 Molec. Cell. Biol. 9: 2847-2853), or CD5 (Jones, N. H. et al., 1986 Nature 323:346-349), or the signal peptide from any extracellular protein.

The mutant molecule can include the oncostatin M signal peptide linked at the N-terminal end of the extracellular domain of CTLA4, and the human immunoglobulin molecule (e.g., hinge, CH2 and CH3) linked to the C-terminal end of the extracellular domain of CTLA4. This preferred molecule includes the oncostatin M signal peptide encompassing methionine at position −26 through alanine at position −1, the CTLA4 portion encompassing methionine at position +1 through aspartic acid at position +124, a junction amino acid residue glutamine at position +125, and the immunoglobulin portion encompassing glutamic acid at position +126 through lysine at position +357.

The soluble CTLA4 mutant molecule can be isolated by molecular or chemical synthesis methods. The molecular methods may include the following steps: introducing a suitable host cell with a nucleic acid molecule that expresses and encodes the soluble CTLA4 mutant molecule; culturing the host cell so introduced under conditions that permit the host cell to express the mutant molecules; and isolating the expressed mutant molecules. The signal peptide portion of the mutant molecule permits the expressed protein molecules to be secreted by the host cell. The secreted mutant molecules can undergo post-translational modification, involving cleavage of the signal peptide to produce a mature protein having the CTLA4 and the immunoglobulin portions. The cleavage may occur after the alanine at position −1, resulting in a mature mutant molecule having methionine at position +1 as the first amino acid (FIG. 7 or 8). Alternatively, the cleavage may occur after the methionine at position −2, resulting in a mature mutant molecule having alanine at position −1 as the first amino acid.

A preferred embodiment is a soluble CTLA4 mutant molecule having the extracellular domain of human CTLA4 linked to the human immunoglobulin molecule (e.g., hinge, CH2 and CH3). This preferred molecule includes the CTLA4 portion encompassing methionine at position +1 through aspartic acid at position +124, a junction amino acid residue glutamine at position +125, and the immunoglobulin portion encompassing glutamic acid at position +126 through lysine at position +357. The portion having the extracellular domain of CTLA4 is mutated so that alanine at position +29 is substituted with tyrosine and leucine at position +104 is substituted with glutamic acid. The immunoglobulin portion of the mutant molecule can be mutated, so that the cysteines at positions +130, +136, and +139 are substituted with serine, and the proline at position +148 is substituted with serine. This mutant molecule is designated herein as L104EA29YIg (FIG. 7).

Alternatively, a preferred embodiment of L104EA29YIg is a mutant molecule having alanine at position −1 through aspartic acid at position +124, a junction amino acid residue glutamine at position +125, and the immunoglobulin portion encompassing glutamic acid at position +126 (e.g., +126 through lysine at position +357). The portion having the extracellular domain of CTLA4 is mutated so that alanine at position +29 is replaced with tyrosine; and leucine at position +104 is replaced with glutamic acid. The immunoglobulin portion of the mutant molecule is mutated so that the cysteines at positions +130, +136, and +139 are replaced with serine, and the proline at position +148 is replaced with serine. This mutant molecule is designated herein as L104EA29YIg (FIG. 7).

Another preferred mutant molecule is a soluble CTLA4 mutant molecule having the extracellular domain of human CTLA4 linked to the human immunoglobulin molecule (e.g., hinge, CH2 and CH3). This preferred molecule includes the CTLA4 portion encompassing methionine at position +1 through aspartic acid at position +124, a junction amino acid residue glutamine at position +125, and the immunoglobulin portion encompassing glutamic acid at position +126 through lysine at position +357. The portion having the extracellular domain of CTLA4 is mutated so that leucine at position +104 is substituted with glutamic acid. The hinge portion of the mutant molecule is mutated so that the cysteines at positions +130, +136, and +139 are substituted with serine, and the proline at position +148 is substituted with serine. This mutant molecule is designated herein as L104EIg (FIG. 8).

Alternatively, the preferred embodiment of L104EIg is a soluble CTLA4 mutant molecule having an extracellular domain of human CTLA4 linked to a human immunoglobulin molecule (e.g., hinge, CH2 and CH3). This preferred molecule includes the CTLA4 portion encompassing alanine at position −1 through aspartic acid at position +124, a junction amino acid residue glutamine at position +125, and the immunoglobulin portion encompassing glutamic acid at position +126 through lysine at position +357. The portion having the extracellular domain of CTLA4 is mutated so that leucine at position +104 is substituted with glutamic acid. The hinge portion of the mutant molecule is mutated so that the cysteines at positions +130, +136, and +139 are substituted with serine, and the proline at position +148 is substituted with serine. This mutant molecule is designated herein as L104EIg (FIG. 8).

Further, the invention provides a soluble CTLA4 mutant molecule having: (a) a first amino acid sequence of a membrane glycoprotein, e.g., CD28, CD86, CD80, CD40, and gp39, which blocks T cell proliferation fused to a second amino acid sequence; (b) the second amino acid sequence being a fragment of the extracellular domain of mutant CTLA4 which blocks T cell proliferation, such as, for example comprising methionine at position +1 through aspartic acid at position +124 (FIG. 7 or 8); and (c) a third amino acid sequence which acts as an identification tag or enhances solubility of the molecule. For example, the third amino acid sequence can consist essentially of amino acid residues of the hinge, CH2 and CH3 regions of a non-immunogenic immunoglobulin molecule. Examples of suitable immunoglobulin molecules include but are not limited to human or monkey immunoglobulin, e.g., C(gamma)1. Other isotypes are possible.

The invention further provides nucleic acid molecules comprising nucleotide sequences encoding the amino acid sequences corresponding to the soluble CTLA4 mutant molecules of the invention. In one embodiment, the nucleic acid molecule is a DNA (e.g., cDNA) or a hybrid thereof. Alternatively, the nucleic acid molecules are RNA or a hybrid thereof.

Additionally, the invention provides a vector which comprises the nucleotide sequences of the invention. A host vector system is also provided. The host vector system comprises the vector of the invention in a suitable host cell. Examples of suitable host cells include but are not limited to prokaryotic and eukaryotic cells.

The invention further provides methods for producing a protein comprising growing the host vector system of the invention so as to produce the protein in the host and recovering the protein so produced.

Additionally, the invention provides methods for regulating functional CTLA4- and CD28-positive T cell interactions with CD80- and/or CD86-positive cells. The methods comprise contacting the CD80- and/or CD86-positive cells with a soluble CTLA4 mutant molecule of the invention so as to form mutant CTLA4/CD80 and/or mutant CTLA4/CD86 complexes, the complexes interfering with reaction of endogenous CTLA4 antigen with CD80 and/or CD86, and/or the complexes interfering with reaction of CD28 antigen with CD80 and/or CD86. In one embodiment of the invention, the soluble CTLA4 mutant molecule is a fusion protein that contains at least a portion of the extracellular domain of mutant CTLA4. In another embodiment, the soluble CTLA4 mutant molecule comprises: a first amino acid sequence including the extracellular domain of CTLA4 from methionine at position +1 to aspartic acid at position +124, including at least one mutation; and a second amino acid sequence including the hinge, CH2, and CH3 regions of the human immunoglobulin gamma 1 molecule (FIG. 7 or 8).

In accordance with the practice of the invention, the CD80- or CD86-positive cells are contacted with fragments or derivatives of the soluble CTLA4 mutant molecules of the invention. Alternatively, the soluble CTLA4 mutant molecule is a CD28Ig/CTLA4Ig fusion protein having a first amino acid sequence corresponding to a portion of the extracellular domain of CD28 receptor fused to a second amino acid sequence corresponding to a portion of the extracellular domain of CTLA4 mutant receptor and a third amino acid sequence corresponding to the hinge, CH2 and CH3 regions of human immunoglobulin C-gamma-1.

The present invention further provides a method for treating immune system diseases mediated by CD28- and/or CTLA4-positive cell interactions with CD80/CD86-positive cells. In one embodiment, T cell interactions are inhibited. This method comprises administering to a subject the soluble CTLA4 mutant molecules of the invention to regulate T cell interactions with the CD80- and/or CD86-positive cells. Alternatively, a CTLA4 mutant hybrid having a membrane glycoprotein joined to CTLA4 mutant molecule can be administered.

The present invention also provides method for inhibiting graft versus host disease in a subject. This method comprises administering to the subject a soluble CTLA4 mutant molecule of the invention together with a ligand reactive with IL-4.

The invention encompasses the use of the soluble CTLA4 mutant molecules together with other immunosuppressants, e.g., cyclosporin (Mathiesen, in: “Prolonged Survival and Vascularization of Xenografted Human Glioblastoma Cells in the Central Nervous System of Cyclosporin A-Treated Rats” 1989 Cancer Lett., 44:151-156), prednisone, azathioprine, and methotrexate (R. Handschumacher “Chapter 53: Drugs Used for Immunosuppression” pages 1264-1276). Other immunosuppressants are possible.

Methods for Producing the Molecules of the Invention

Expression of CTLA4 mutant molecules in prokaryotic cells is preferred for some purposes. Prokaryotes most frequently are represented by various strains of bacteria. The bacteria may be a gram positive or a gram negative. Typically, gram-negative bacteria such as E. coli are preferred. Other microbial strains may also be used.

Sequences encoding CTLA4 mutant molecules can be inserted into a vector designed for expressing foreign sequences in prokaryotic cells such as E. coli. These vectors can include commonly used prokaryotic control sequences which are defined herein to include promoters for transcription initiation, optionally with an operator, along with ribosome binding site sequences, include such commonly used promoters as the beta-lactamase (penicillinase) and lactose (lac) promoter systems (Chang, et al., 1977 Nature 198:1056), the tryptophan (trp) promoter system (Goeddel, et al., 1980 Nucleic Acids Res. 8:4057) and the lambda derived PL promoter and N-gene ribosome binding site (Shimatake, et al., 1981 Nature 292:128).

Such vectors will also include origins of replication and selectable markers, such as a beta-lactamase or neomycin phosphotransferase gene conferring resistance to antibiotics so that the vectors can replicate in bacteria and cells carrying the plasmids can be selected for when grown in the presence of ampicillin or kanamycin.

The expression plasmid can be introduced into prokaryotic cells via a variety of standard methods, including but not limited to CaCl₂-shock (Cohen, 1972 Proc. Natl. Acad. Sci. USA 69:2110, and Sambrook et al. (eds.), “Molecular Cloning: A Laboratory Manual”, 2nd Edition, Cold Spring Harbor Press, (1989)) and electroporation.

In accordance with the practice of the invention, eukaryotic cells are also suitable host cells. Examples of eukaryotic cells include any animal cell, whether primary or immortalized, yeast (e.g., Saccharomyces cerevisiae, Schizosaccharomyces pombe, and Pichia pastoris), and plant cells. Myeloma, COS and CHO cells are examples of animal cells that may be used as hosts. Exemplary plant cells include tobacco (whole plants or tobacco callus), corn, soybean, and rice cells. Corn, soybean, and rice seeds are also acceptable.

Sequences encoding the CTLA4 mutant molecules can be inserted into a vector designed for expressing foreign sequences in a eukaryotic host. The regulatory elements of the vector can vary according to the particular eukaryotic host.

Commonly used eukaryotic control sequences include promoters and control sequences compatible with mammalian cells such as, for example, CMV promoter (CDM8 vector) and avian sarcoma virus (ASV) (πLN vector). Other commonly used promoters include the early and late promoters from Simian Virus 40 (SV40) (Fiers, et al., 1973 Nature 273:113), or other viral promoters such as those derived from polyoma, Adenovirus 2, and bovine papilloma virus. An inducible promoter, such as hMTII (Karin, et al., 1982 Nature 299:797-802) may also be used.

Vectors for expressing CTLA4 mutant molecules in eukaryotes may also carry sequences called enhancer regions. These are important in optimizing gene expression and are found either upstream or downstream of the promoter region.

Sequences encoding CTLA4 mutant molecules can integrate into the genome of the eukaryotic host cell and replicate as the host genome replicates. Alternatively, the vector carrying CTLA4 mutant molecules can contain origins of replication allowing for extrachromosomal replication.

For expressing the sequences in Saccharomyces cerevisiae, the origin of replication from the endogenous yeast plasmid, the 2μ circle could be used. (Broach, 1983 Meth. Enz. 101:307). Alternatively, sequences from the yeast genome capable of promoting autonomous replication could be used (see, for example, Stinchcomb et al., 1979 Nature 282:39); Tschemper et al., 1980 Gene 10:157; and Clarke et al., 1983 Meth. Enz. 101:300).

Transcriptional control sequences for yeast vectors include promoters for the synthesis of glycolytic enzymes (Hess et al., 1968 J. Adv. Enzyme Reg. 7:149; Holland et al., 1978 Biochemistry 17:4900). Additional promoters known in the art include the CMV promoter provided in the CDM8 vector (Toyama and Okayama, 1990 FEBS 268:217-221); the promoter for 3-phosphoglycerate kinase (Hitzeman et al., 1980 J. Biol. Chem. 255:2073), and those for other glycolytic enzymes.

Other promoters are inducible because they can be regulated by environmental stimuli or the growth medium of the cells. These inducible promoters include those from the genes for heat shock proteins, alcohol dehydrogenase 2, isocytochrome C, acid phosphatase, enzymes associated with nitrogen catabolism, and enzymes responsible for maltose and galactose utilization.

Regulatory sequences may also be placed at the 3′ end of the coding sequences. These sequences may act to stabilize messenger RNA. Such terminators are found in the 3′ untranslated region following the coding sequences in several yeast-derived and mammalian genes.

Exemplary vectors for plants and plant cells include but are not limited to Agrobacterium Tᵢ plasmids, cauliflower mosaic virus (CaMV), tomato golden mosaic virus (TGMV).

General aspects of mammalian cell host system transformations have been described by Axel (U.S. Pat. No. 4,399,216 issued Aug. 16, 1983). Mammalian cells be transformed by methods including but not limited to, transfection in the presence of calcium phosphate, microinjection, electroporation, or via transduction with viral vectors.

Methods for introducing foreign DNA sequences into plant and yeast genomes include (1) mechanical methods, such as microinjection of DNA into single cells or protoplasts, vortexing cells with glass beads in the presence of DNA, or shooting DNA-coated tungsten or gold spheres into cells or protoplasts; (2) introducing DNA by making protoplasts permeable to macromolecules through polyethylene glycol treatment or subjection to high voltage electrical pulses (electroporation); or (3) the use of liposomes (containing cDNA) which fuse to protoplasts.

Expression of CTLA4 mutant molecules can be detected by methods known in the art. For example, the mutant molecules can be detected by Coomassie staining SDS-PAGE gels and immunoblotting using antibodies that bind CTLA4. Protein recovery can be performed using standard protein purification means, e.g., affinity chromatography or ion-exchange chromatography, to yield substantially pure product (R. Scopes in: “Protein Purification, Principles and Practice”, Third Edition, Springer-Verlag 1994).

CTLA4Ig Codon-Based Mutagenesis

In one embodiment, site-directed mutagenesis and a novel screening procedure were used to identify several mutations in the extracellular domain of CTLA4 that improve binding avidity for CD86, while only marginally altering binding to CD80. In this embodiment, mutations were carried out in residues in serine 25 to arginine 33, the C′ strand (alanine 49 and threonine 51), the F strand (lysine 93, glutamic acid 95 and leucine 96), and in methionine 97 through tyrosine 102, tyrosine 103 through glycine 107 and in the G strand at positions glutamine 111, tyrosine 113 and isoleucine 115. These sites were chosen based on studies of chimeric CD28/CTLA4 fusion proteins (J. Exp. Med., 1994, 180:2049-2058), and on a model predicting which amino acid residue side chains would be solvent exposed, and a lack of amino acid residue identity or homology at certain positions between CD28 and CTLA4. Also, any residue which is spatially in close proximity (5 to 20 Angstrom Units) to the identified residues are considered part of the present invention.

To synthesize and screen soluble CTLA4 mutant molecules with altered affinities for CD86, a two-step strategy was adopted. The experiments entailed first generating a library of mutations at a specific codon of an extracellular portion of CTLA4 and then screening these by BIAcore analysis to identify mutants with altered reactivity to CD80 or CD86.

Advantages of the Present Invention

Soluble CTLA4 mutant molecules having a higher avidity for CD80- or CD86-positive cells compared to wild type CTLA4 or non-mutated forms of CTLA4Ig are expected to block the priming of antigen specific activated cells with higher efficiency than wild type CTLA4 or non-mutated forms of CTLA4Ig.

Further, production costs for CTLA4Ig are very high. The high avidity mutant CTLA4Ig molecules having higher potent immunosuppressive properties could be used in the clinic at considerably lower doses than non-mutated CTLA4Ig to achieve similar levels of immunosuppression. Soluble CTLA4 mutant molecules, e.g., L104EA29YIg, could be very cost effective.

The following example is presented to illustrate the present invention and to assist one of ordinary skill in making and using the same. This example is not intended in any way to otherwise limit the scope of the invention.

### EXAMPLE 1

The following provides a description of the methods used to generate the nucleotide sequences encoding the soluble CTLA4 mutant molecules of the invention. A single-site mutant L104EIg was generated and tested for binding kinetics for CD80 and/or CD86. The L104EIg nucleotide sequence was used as a template to generate the double-site mutant CTLA4 sequence, L104EA29YIg, which was tested for binding kinetics.

CTLA4Ig Codon Based Mutagenesis:

Single-site mutant nucleotide sequences were generated using CTLA4Ig (U.S. Pat. Nos. 5,844,095; 5,851,795; and 5,885,796) as a template. Mutagenic oligonucleotide PCR primers were designed for random mutagenesis of a specific codon by allowing any base at positions 1 and 2 of the codon, but only guanine or thymine at position 3 (XXG/T). In this manner, a specific codon encoding an amino acid could be randomly mutated to code for each of the 20 amino acids. PCR products encoding mutations in close proximity to -M97-G107 of CTLA4Ig (see FIG. 7 or 8), were digested with SacI/XbaI and subcloned into similarly cut CTLA4Ig πLN expression vector. This method was used to generate the single-site CTLA4 mutant molecule L104EIg (FIG. 8).

For mutagenesis in proximity to S25-R33 of CTLA4Ig, a silent NheI restriction site was first introduced 5′ to this loop, by PCR primer-directed mutagenesis. PCR products were digested with NheI/XbaI and subcloned into similarly cut CTLA4Ig or L104EIg expression vectors. This method was used to generate the double-site CTLA4 mutant molecule L104EA29YIg (FIG. 7).

### EXAMPLE 2

The following provides a description of the screening methods used to identify the single- and double-site mutant CTLA polypeptides, expressed from the constructs described in Example 1, that exhibited a higher binding avidity for CD80 and CD86 antigens, compared to non-mutated CTLA4Ig molecules.

Current in vitro and in vivo studies indicate that CTLA4Ig by itself is unable to completely block the priming of antigen specific activated T cells. In vitro studies with CTLA4Ig and either monoclonal antibody specific for CD80 or CD86 measuring inhibition of T cell proliferation indicate that anti-CD80 monoclonal antibody did not augment CTLA4Ig inhibition. However, anti-CD86 monoclonal antibody did, indicating that CTLA4Ig was not as effective at blocking CD86 interactions. These data support earlier findings by Linsley et al. (Immunity, 1994, 1:793-801) showing inhibition of CD80-mediated cellular responses required approximately 100 fold lower CTLA4Ig concentrations than for CD86-mediated responses. Based on these findings, it was surmised that soluble CTLA4 mutant molecules having a higher avidity for CD86 than wild type CTLA4 should be better able to block the priming of antigen specific activated cells than CTLA4Ig.

To this end, the soluble CTLA4 mutant molecules described in Example 1 above were screened using a novel screening procedure to identify several mutations in the extracellular domain of CTLA4 that improve binding avidity for CD80 and CD86.

In general, COS cells were transfected with individual miniprep plasmid cDNA and three day conditioned culture media applied to BIAcore biosensor chips (Pharmacia Biotech AB, Uppsala, Sweden) coated with soluble CD80Ig or CD86Ig. The specific binding and dissociation of mutant proteins was measured by surface plasmon resonance (O'Shannessy, D. J., et al., 1997 Anal. Biochem. 212:457-468).

Screening Method

COS cells grown in 24 well tissue culture plates were transiently transfected with mutant CTLA4Ig and culture media collected 3 days later.

Conditioned COS cell culture media was allowed to flow over BIAcore biosensor chips derivatized with CD86 μg or CD80Ig, and mutant molecules were identified with off rates slower than that observed for wild type CTLA4Ig. The cDNAs corresponding to selected media samples were sequenced and DNA was prepared from these cDNAs to perform larger scale COS cell transient transfection, from which mutant CTLA4Ig protein was prepared following protein A purification of culture media.

BIAcore analysis conditions and equilibrium binding data analysis were performed as described in J. Greene et al. 1996 J. Biol. Chem. 271:26762.

BIAcore Data Analysis

Senosorgram baselines were normalized to zero response units (RU) prior to analysis. Samples were run over mock-derivatized flow cells to determine background RU values due to bulk refractive index differences between solutions. Equilibrium dissociation constants (K_{d}) were calculated from plots of R_{eq} versus C, where R_{eq} is the steady-state response minus the response on a mock-derivatized chip, and C is the molar concentration of analyte. Binding curves were analyzed using commercial nonlinear curve-fitting software (Prism, GraphPAD Software).

Experimental data were first fit to a model for a single ligand binding to a single receptor (1-site model, i.e., a simple langmuir system, A+B→AB), and equilibrium association constants (K_{d}=[A]·[B]\[AB]) were calculated from the equation R=Rₘₐₓ·C/(K_{d}+C). Subsequently, data were fit to the simplest two-site model of ligand binding (i.e., to a receptor having two non-interacting independent binding sites as described by the equation R=Rₘₐₓ₁·C\(K_{d1}+C)+Rₘₐₓ₂·C\(K_{d2}+C).

The goodness-of-fits of these two models were analyzed visually by comparison with experimental data and statistically by an F test of the sums-of-squares. The simpler one-site model was chosen as the best fit unless the two-site model fit significantly better (p<0.1).

Association and disassociation analyses were performed using BIA evaluation 2.1 Software (Pharmacia). Association rate constants kₒₙ were calculated in two ways, assuming both homogenous single-site interactions and parallel two-site interactions. For single-site interactions, kₒₙ values were calculated according to the equation Rₜ=R_{eq}(1−exp^{-ks(t-t}₀), where Rₜ is a response at a given time, t; R_{eq} is the steady-state response; to is the time at the start of the injection; and kₛ=dR/dt=kₒₙ·C_{off}, where C is a concentration of analyte, calculated in terms of monomeric binding sites. For two-site interactions kₒₙ values were calculated according to the equation Rₜ=R_{eq1}(1−exp^{-ks1(t-t}₀)+R_{eq2}(1−exp^{ks2(t-t}₀). For each model, the values of kₒₙ were determined from the calculated slope (to about 70% maximal association) of plots of kₛ versus C.

Dissociation data were analyzed according to one site (AB=A+B) or two sites (AiBj=Ai+Bj) models, and rate constants (k_{off}) were calculated from best fit curves. The binding site model was used except when the residuals were greater than machine background (2-10RU, according to machine), in which case the two-binding site model was employed. Half-times of receptor occupancy were calculated using the relationship t_{1/2}=0.693/k_{off}.

Flow Cytometry:

Murine MAb L307.4 (anti-CD80) was purchased from Becton Dickinson (San Jose, Calif.) and IT2.2 (anti-B7-0 [also known as CD86]), from Pharmingen (San Diego, Calif.). For immunostaining, CD80 and/or CD86+ CHO cells were removed from their culture vessels by incubation in phosphate-buffered saline containing 100 mM EDTA. CHO cells (1-10×10⁵) were first incubated with MAbs or immunoglobulin fusion proteins in DMEM containing 10% fetal bovine serum (FBS), then washed and incubated with fluorescein isothiocyanate-conjugated goat anti-mouse or anti-human immunoglobulin second step reagents (Tago, Burlingame, Calif.). Cells were given a final wash and analyzed on a FACScan (Becton Dickinson).

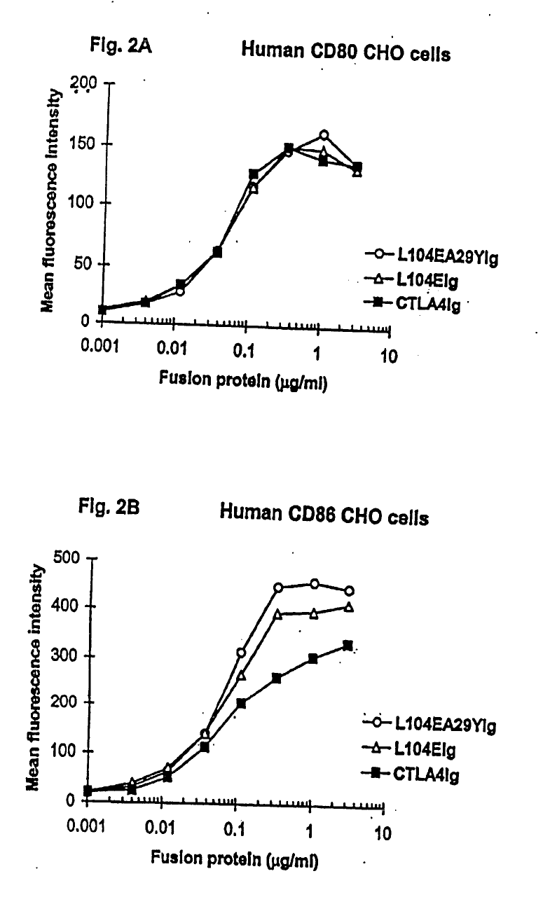

FACS analysis (FIG. 2) of CTLA4Ig and mutant molecules binding to stably transfected CD80+ and CD86+ CHO cells was performed as described herein.

CD80+ and CD86+ CHO cells were incubated with increasing concentrations of CTLA4Ig, washed and bound immunoglobulin fusion protein was detected using fluorescein isothiocyanate-conjugated goat anti-human immunoglobulin.

In FIG. 2, L104EA29YIg (circles), or L104EIg (triangle) CHO cells (1.5×10⁵) were incubated with the indicated concentrations of CTLA4Ig (closed square), L104EA29YIg (circles), or L104EIg (triangle) for 2 hr. at 23° C., washed, and incubated with fluorescein isothiocyanate-conjugated goat anti-human immunoglobulin antibody. Binding on a total of 5,000 viable cells was analyzed (single determination) on a FACScan, and mean fluorescence intensity (MFI) was determined from data histograms using PC-LYSYS. Data have been corrected for background fluorescence measured on cells incubated with second step reagent only (MFI=7). Control L6 MAb (80 μg/ml) gave MFI<30. This is representative of four independent experiments.

Functional Assays:

Human CD4⁺T cells were isolated by immunomagnetic negative selection (Linsley et al., 1992 J. Exp. Med. 176:1595-1604).

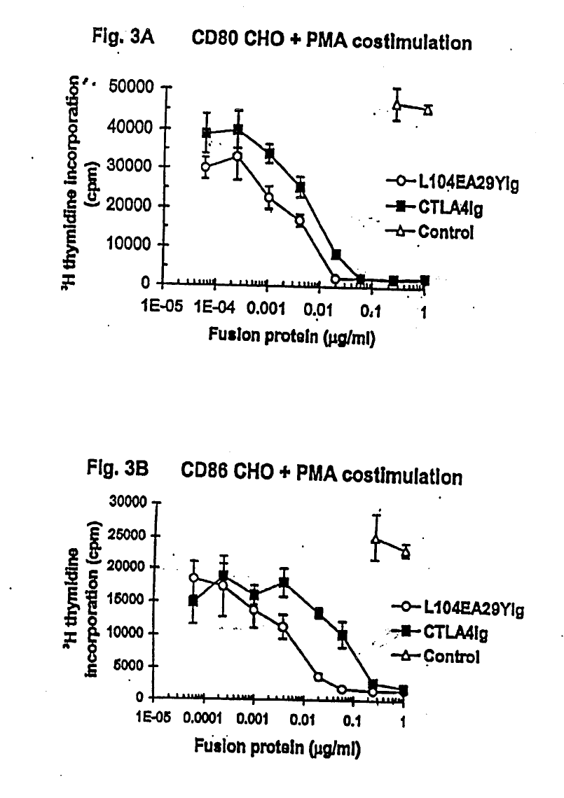

Inhibition of PMA plus CD80-CH0 or CD86-CHO T cell stimulation (FIG. 3) was performed. CD4⁺T cells (8-10×10⁴/well) were cultured in the presence of 1 nM PMA with or without irradiated CHO cell stimulators. Proliferative responses were measured by the addition of 1 μCi/well of [³H]thymidine during the final 7 hr. of a 72 hr. culture.

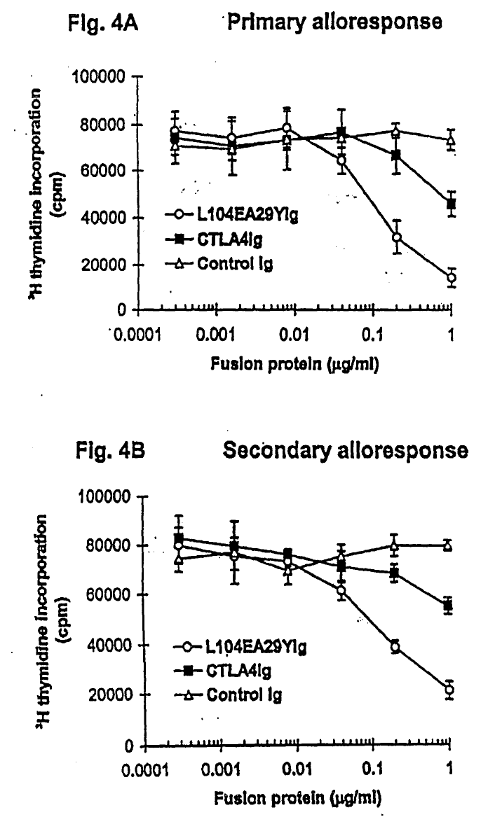

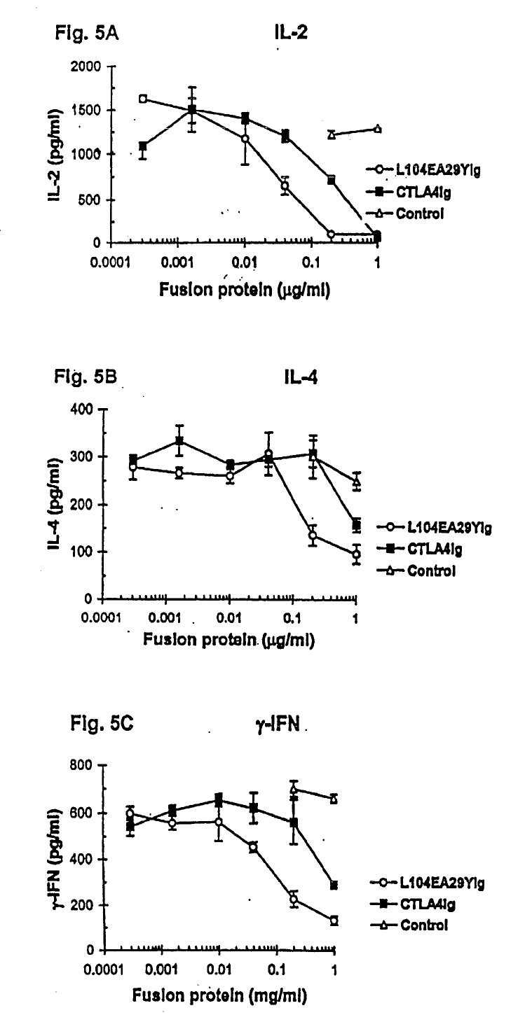

FIGS. 4 and 5 show inhibition of allostimulated human T cells prepared above, and allostimulated with a human B LCL line called PM. T cells at 3.0×10⁴/well and PM at 8.0×10³/well. Primary allostimulation occurred for 6 days then the cells were pulsed with ³H-thymidine for 7 hours before incorporation of radiolabel was determined. Secondary allostimulation was performed as follows. Seven day primary allostimulated T cells were harvested over LSM (Ficol) and rested for 24 hours. T cells then restimulated (secondary) by adding PM in same ratio as above. Stimulation occurred for 3 days, then the cells were pulsed with radiolabel and harvested as above. To measure cytokine production (FIG. 5), duplicate secondary allostimulation plates were set up. After 3 days, culture media was assayed using Biosource kits using conditions recommended by manufacturer.

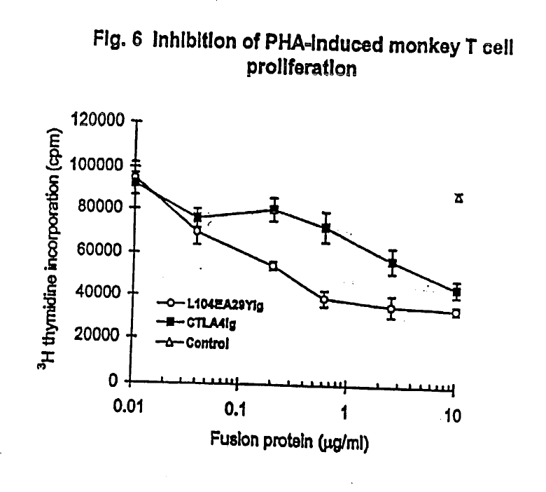

Monkey MLR (FIG. 6). PBMC'S from 2 monkeys purified over LSM and mixed (3.5×10⁴ cells/well from each monkey) with 2 ug/ml PHA. Stimulated 3 days then pulsed with radiolabel 16 hours before harvesting.

TABLE I
Equilibrium binding constants

| | CD80Ig (Kd) | CD86Ig (Kd) |
|---|---|---|
| CTLA4Ig | 6.51 ± 1.08 | 13.9 ± 2.27 |
| L104EIg | 4.47 ± 0.36 | 6.06 ± 0.05 |
| L104EA29YIg | 3.66 ± 0.41 | 3.21 ± 0.23 |

BIAcore™ Analysis: All experiments were run on BIAcore™ or BIAcore™ 2000 biosensors (Pharmacia Biotech AB, Uppsala) at 25° C. Ligands were immobilized on research grade NCM5 sensor chips (Pharmacia) using standard N-ethyl-N′-(dimethylaminopropyl) carbodiimidN-hydroxysuccinimide coupling (Johnsson, B., et al. 1991 Anal. Biochem. 198: 268-277; Khilko, S. N., et al. 1993 J. Biol. Chem 268:5425-15434).

What is claimed is:

1. A soluble CTLA4 mutant molecule which binds CD80 and/or CD86 comprising an extracellular domain of CTLA4 so that (a) an alanine at position +29 is substituted with an amino acid selected from the group consisting of tyrosine, leucine, phenylalanine, tryptophan, and threonine, and (b) a leucine at position +104 is substituted with a glutamic acid.

2. The soluble CTLA4 mutant molecule of claim 1 further comprising an amino acid sequence which alters the solubility, affinity or valency of the soluble CTLA4 mutant molecule for binding to the CD80 and/or CD86 molecule.

3. The soluble CTLA4 mutant molecule of claim 2, wherein the amino acid sequence comprises a human immunoglobulin constant region.

4. The soluble CTLA4 mutant molecule of claim 2 further comprising an amino acid sequence which permits secretion of the soluble CTLA4 mutant molecule.

5. The soluble CTLA4 mutant molecule of claim 4, wherein the amino acid sequence comprises an oncostatin M signal peptide.

6. The soluble CTLA4 mutant molecule of claim 1 comprising methionine at position +1 through aspartic acid at position +124 as shown in FIG. 7.

7. The soluble CTLA4 mutant molecule of claim 1, comprising alanine at position −1 through aspartic acid at position +124 as shown in FIG. 7.

8. The soluble CTLA4 mutant molecule of claim 3, wherein the human immunoglobulin constant region is mutated to include a cysteine at position +130 substituted with a serine, a cysteine at position +136 substituted with a serine, a cysteine at position +139 substituted with a serine, and a proline at position +148 substituted with serine, as shown in FIG. 7.

9. A soluble CTLA4 mutant molecule which binds with higher avidity to CD80 and/or CD86 than CTLA4, comprising an extracellular domain of CTLA4, wherein in the extracellular domain, alanine at position +29 is substituted with tyrosine and leucine at position +104 is substituted with glutamic acid as shown in FIG. 7.

10. A soluble CTLA4 mutant molecule which binds with higher avidity to the CD80 and/or CD86 than CTLA4, comprising an extracellular domain of CTLA4, wherein in the extracellular domain, leucine at position +104 is substituted with glutamic acid as shown in FIG. 8.

11. A nucleic acid molecule comprising a nucleotide sequence encoding the amino acid sequence corresponding to the soluble CTLA4 mutant molecule of claim 1.

12. The nucleic acid molecule of claim 11 having the sequence beginning with adenine at nucleotide position +1 and ending with adenine at +1071 as shown in FIG. 7 or 8.

13. The nucleic acid molecule of claim 11 having the sequence beginning with guanidine at −3 and ending at adenine at +1071 as shown in FIG. 7 or 8.

14. A vector comprising the nucleotide sequence of claim 11.

15. A host vector system comprising a vector of claim 14 in a suitable host cell.

16. The host vector system of claim 15, wherein the suitable host cell is a bacterial cell or a eukaryotic cell.

17. A method for producing a soluble CTLA mutant protein comprising growing the host vector system of claim 15 so as to produce the protein in the host cell and recovering the protein so produced.

18. A soluble CTLA mutant protein produced by the method of claim 17.

19. A method for regulating a T cell interaction with a CD80 and/or CD86 positive cell comprising contacting the CD80 and/or CD86 positive cell with the soluble CTLA4 mutant molecule of claim 1 so as to form a CTLA4 mutant molecule/CD80 or a CTLA4 mutant molecule/CD86 complex, the complex interfering with interaction between the T cell and the CD80 and/or CD86 positive cell.

20. The method of claim 20, wherein the soluble CTLA4 mutant comprises an extracellular domain of CTLA4, wherein in the extracellular domain, alanine at position +29 is substituted with tyrosine and leucine at position +104 is substituted with glutamic acid as shown in FIG. 7.

21. The method of claim 20, wherein the soluble CTLA4 mutant molecule comprises an extracellular domain of CTLA4, wherein in the extracellular domain, leucine at position +104 is substituted with glutamic acid as shown in FIG. 8.

22. The method of claim 20, wherein the CD80 and/or CD86 positive cell is contacted with a fragment or a derivative of the soluble CTLA4 mutant molecule.

23. The method of claim 20, wherein the CD80 and/or CD86 positive cell is an APC cell.

24. The method of claim 20, wherein the interaction of the CTLA4-positive T cells with the CD80 and CD86 positive cells is inhibited.

25. A method for treating immune system diseases mediated by T cell interactions with CD80 and/or CD86 positive cells comprising administering to a subject the soluble CTLA4 mutant molecule of claim 1 to regulate T cell interactions with the CD86 positive cells.

26. The method of claim 25, wherein the soluble CTLA4 mutant molecule comprises an extracellular domain of CTLA4, wherein in the extracellular domain, alanine at position +29 is substituted with tyrosine and leucine at position +104 is substituted with glutamic acid as shown in FIG. 7.

27. The method of claim 25, wherein the soluble CTLA4 mutant comprises an extracellular domain of CTLA4, wherein in the extracellular domain, alanine at position +29 is substituted with tyrosine and leucine at position +104 is substituted with glutamic acid as shown in FIG. 7.

28. The method of claim 25, wherein said T cell interactions are inhibited.

29. A method for inhibiting graft versus host disease in a subject which comprises administering to the subject the soluble CTLA4 mutant molecule of claim 1 and a ligand reactive with IL-4.

30. The method of claim 29, wherein the soluble CTLA4 mutant molecule comprises an extracellular domain of CTLA4, wherein in the extracellular domain, alanine at position +29 is substituted with tyrosine and leucine at position +104 is substituted with glutamic acid as shown in FIG. 7.

31. The method of claim 29, wherein the soluble CTLA4 mutant comprises an extracellular domain of CTLA4, wherein in the extracellular domain, alanine at position +29 is substituted with tyrosine and leucine at position +104 is substituted with glutamic acid as shown in FIG. 7.

32.
